(19) United States
(12) Patent Application Publication
(10) Pub. No.: US 2012/0116084 A1
(43) Pub. Date: May 10, 2012

(54) N-TYPE ORGANIC SEMICONDUCTORS INCLUDING AT LEAST TWO 2-DICYANOMETHYLENE-3-CYANO-2,5-DIHYDROFURAN GROUPS

(75) Inventors: Clément Suspene, Tulle (FR); Jean-Pierre Simonato, Sassenage (FR)

(73) Assignee: COMMISARIAT A L'ENERGIE ATOMIQUE ET AUX ENE ALT, Paris (FR)

(21) Appl. No.: 13/377,403

(22) PCT Filed: Jun. 3, 2010
(86) PCT No.: PCT/FR2010/000409

§ 371 Date: Jan. 19, 2012

(30) Foreign Application Priority Data
Jun. 9, 2009 (FR) 09 02777

(51) Int. Cl. C07D 407/10; C07D 409/14; C07D 471/06

(52) U.S. Cl. 546/37; 549/472; 549/60

(57) ABSTRACT

The invention relates to a method for manufacturing transistors. Said method involves using a molecule including at least two 2-dicyanomethylene-3-cyano-2,5-dihydrofuran groups. The invention can be implemented in particular in the field of electronics.

The invention relates to a molecule comprising at least two 2-dicyanomethylene-3-cyano-2,5-dihydrofuran groups, and also to the uses thereof.

The use of electronic devices such as computer and television screens, bank cards or new-generation cellphones is omnipresent in our daily life.

Transistors are essential constituents of these devices.

These transistors have been based, since 1947, for the most part on inorganic semiconductors and especially on silicon.

However, technological limitations associated with the use of the latter have recently led to the development of transistors integrating organic semiconductors.

The ease of use of these compounds, which are polymers or small conjugated molecules, gives them interesting potential in the field of low-cost major-retail electronics, although their mobility is less than that of crystalline silicon.

These organic semiconductors may also be used for novel applications such as modern identification means or for electronic display.

n-type organic semiconductors involve the movement of electrons in electronic devices.

However, at the present time, n-type organic semiconductors are not numerous and generally belong to the fullerene family.

Specifically, the majority of the existing organic semiconductors, whether they are polymers or small conjugated molecules, are p-type conductors, i.e. they more easily transport “holes” than electrons.

Now, there is currently growing interest in the development of n-type transistors, especially for the manufacture of complementary circuits.

These n-type materials must have high mobility, sufficiently high electron affinity to ensure effective injection of electrons into the lowest unoccupied molecular orbital (LUMO), and, finally, show little deterioration of their electronic properties over time.

In order to make up for the small number of available n-type semiconductors, a strategy has been envisioned, which consists in inverting the p to n nature of the known p-type semiconductors.

This inversion has been described several times in the literature and involves bonding electron-withdrawing groups to these p-type semiconductors.

These electron-withdrawing groups especially include fluorine atoms, fluorocarbon groups of the type —(CF₂)ₙCF₃, cyano groups, malononitrile groups, anhydride groups or imide groups.

With the introduction of these groups into p-type semiconductor materials, the electrons are “attracted” to these groups, the “core” of the molecules becoming electron-deficient and thus possibly serving for the transportation of the injected negative charges.

The mobilities of devices formed from crystalline films of these compounds can now reach about 1 cm². v⁻¹. s^{—1} in top-gate top-contact geometry, which justifies the growing interest in these materials despite the fact that top-gate top-contact geometry gives mobilities that are very much higher than the mobilities of devices with bottom-gate bottom-contact geometry.

Among the most significant examples of n-type semiconductors prepared by this method, mention may be made of perylene diimides such as PDI-8CN₂, naphthalene diimides or fluorinated quaterthiophenes such as DFCO-4T (M. Mas-Torrent and C. Rovira Chem. Soc. Rev. 2008, 37, 827).

A major problem lies in the fact that the increase in n nature of a molecule is generally concomitant with the decrease of its solubility in organic solvents.

Specifically, during the use of fluorine groups, for example, it arises that the solubility of the compounds obtained is so low that the liquid-route deposition of semiconductors proves to be impossible.

Sublimation then becomes the only possibility for manufacturing devices incorporating crystals or crystalline films of n-type semiconductors, this route moreover being the one most widely used at the present time.

The case of naphthalene diimides bearing perfluorinated alkyl side chains may be mentioned (K. C. See, C. Landis, A. Sarjeant and H. E. Katz Chem. Mater. 2008, 20, 3609).

Furthermore, few of the current n-type semiconductors are air-stable, i.e. their electronic properties generally deteriorate gradually over time.

The few existing examples concern naphthalene or perylene diimides, copper phthalocyanins and thiophenes bearing perfluoro side chains or fluoro groups or cyano groups on the aromatic rings.

The availability of n-type semiconductors that can be manipulated in solution and that are air-stable is thus greatly limited at the present time, PDI-8CN₂ being the most widely used compound that satisfies these criteria.

Consequently, the aim of the invention is to provide molecules with n-type semiconducting properties while at the same time showing good solubility in an organic solvent, and being air-stable, i.e. comprising at least two 2-dicyanomethylene-3-cyano-2,5-dihydrofuran groups bonded to a group bearing conjugated pi bonds comprising at least one 5- or 6-membered aromatic ring.

First and foremost, the invention proposes a process for manufacturing a transistor, characterized in that it comprises a step of using a molecule of formula (I) below:

(GEA)ₙ-R-(DCDHF)ₘ Formula (I)

in which:
GEA denotes an electron-withdrawing group,
n is a positive integer between zero and 10 inclusive,
R is a group bearing conjugated pi bonds comprising at least one inclusively 4-membered to 6-membered aromatic ring, this aromatic ring optionally comprising at least one heteroatom chosen from N, O, P, S, Si and Ge, and being optionally substituted,
DCDHF represents a 2-dicyanomethylene-3-cyano-2,5-dihydrofuran group of formula (II) below:

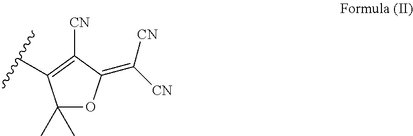

m is an integer between 2 and 10 inclusive.

The invention thus also proposes a process for manufacturing a transistor, characterized in that it comprises a step of depositing, onto at least one surface of a substrate, a solution comprising at least one molecule of formula (I) below:

(GEA)ₙ-R-(DCDHF)ₘ Formula (I)

in which:
GEA denotes an electron-withdrawing group,
n is a positive integer between zero and 10 inclusive,
R is a group bearing conjugated pi bonds comprising at least one inclusively 4-membered to 6-membered aromatic ring, this aromatic ring optionally comprising at least one heteroatom chosen from N, O, P, S, Si and Ge, and being optionally substituted,
DCDHF represents a 2-dicyanomethylene-3-cyano-2,5-dihydrofuran group of formula (II) below:

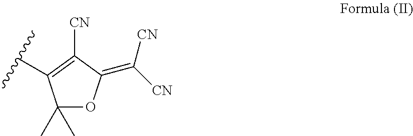

m is an integer between 2 and 10 inclusive.

In the process of the invention, preferably, in the molecule of formula (I), the at least one aromatic ring of the group R is an aromatic ring substituted with unsaturated groups.

Still preferably, the molecule of formula (I) has at least one axis or plane of symmetry, advantageously C₂ symmetry.

Preferably also, in the molecule of formula (I), GEA is chosen from a fluorine atom, a cyano group, a fluorocarbon group, a malononitrile group, an anhydride group, an imide group, a nitro group, a quaternary ammonium group, an ester group, an amide group and a sulfonyl group, and mixtures thereof.

Also preferably, in the molecule of formula (I), n is an integer between 0 and 4 inclusive.

In preferred embodiments, the molecule of formula (I) has one of the formulae 1 to 5 below:

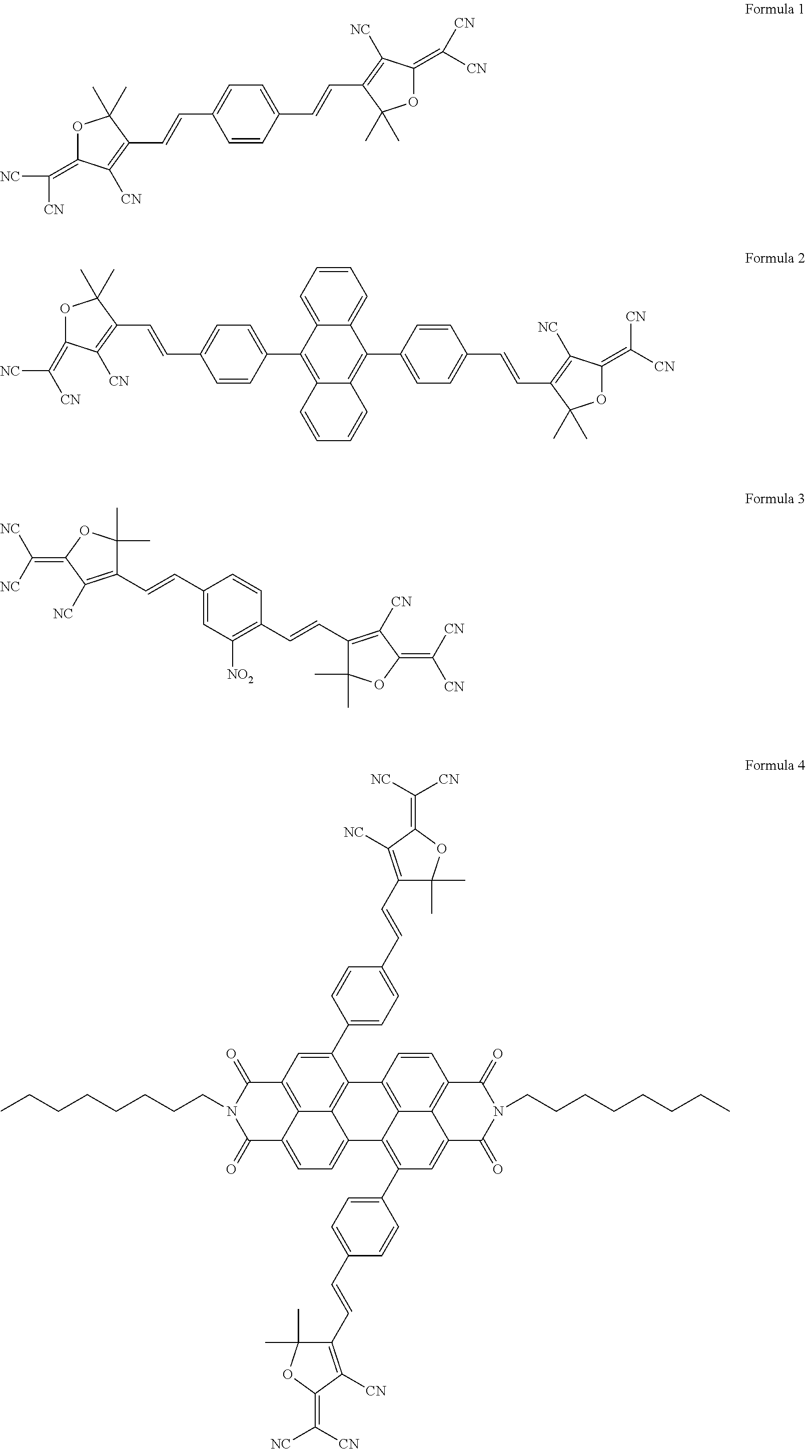

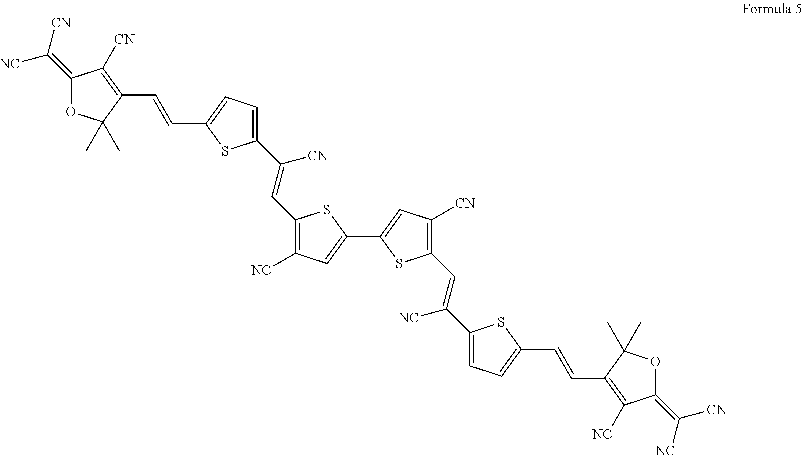

The invention also proposes a molecule, characterized in that it has the formula (I) below:

(GEA)ₙ-R-(DCDHF)ₘ Formula (I)

in which:
GEA denotes an electron-withdrawing group,
n is a positive integer between zero and 10 inclusive,
R is a group bearing conjugated pi bonds comprising at least one inclusively 4-membered to 6-membered aromatic ring, this aromatic ring optionally comprising at least one heteroatom chosen from N, O, P, S, Si and Ge, and being optionally substituted,
DCDHF represents a 2-dicyanomethylene-3-cyano-2,5-dihydrofuran group of formula (II) below:

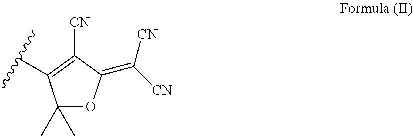

m is an integer between 2 and 10 inclusive, on condition that when n=0, then the molecule has the formula 1 or 2 below:

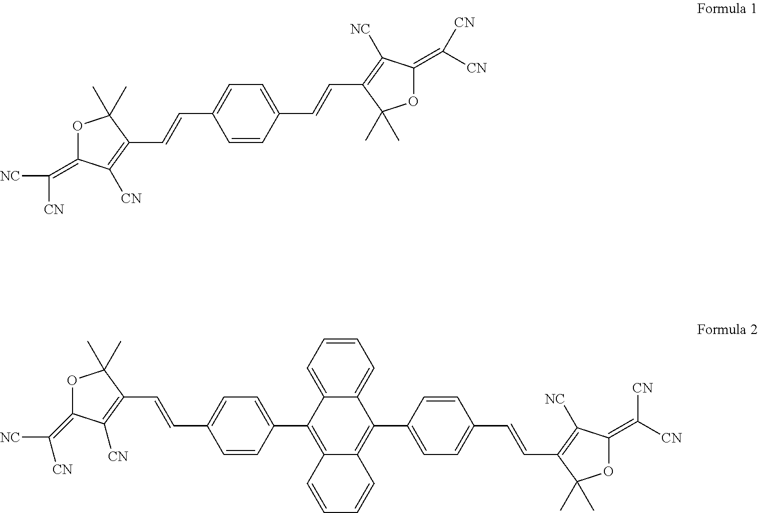

Preferably, this molecule is chosen from the molecules of formulae 1 to 5 below:

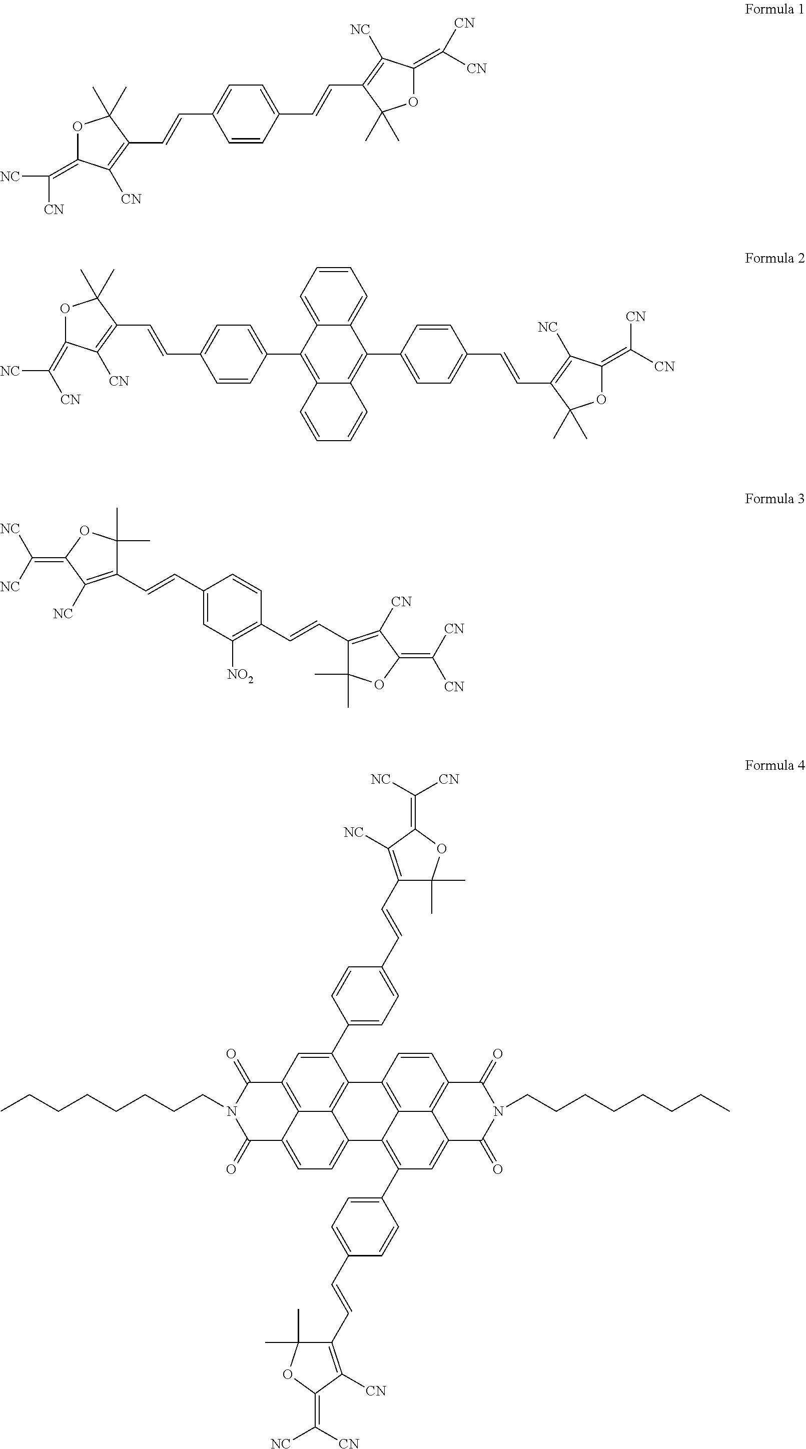

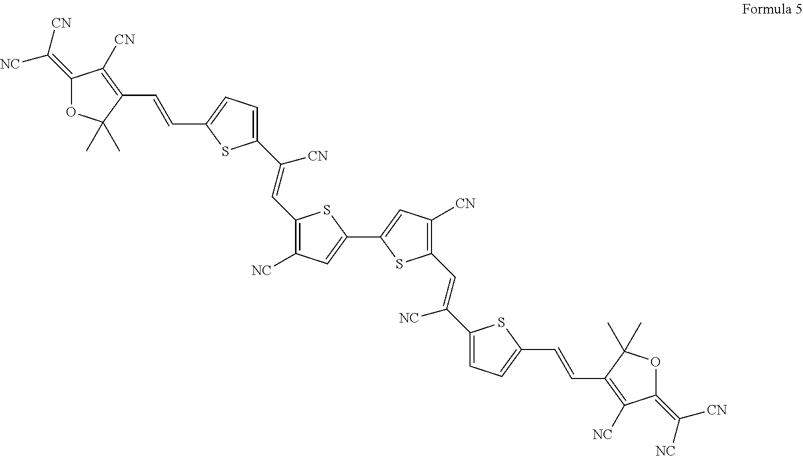

The invention also proposes a transistor, characterized in that it comprises at least one layer comprising at least one molecule of formula (I) below:

(GEA)ₙ-R-(DCDHF)ₘ Formula (I)

in which:
GEA denotes an electron-withdrawing group,
n is a positive integer between zero and 10 inclusive,
R is a group bearing conjugated pi bonds comprising at least one inclusively 4-membered to 6-membered aromatic ring, this aromatic ring optionally comprising at least one heteroatom chosen from N, O, P, S, Si and Ge, and being optionally substituted,
DCDHF represents a 2-dicyanomethylene-3-cyano-2,5-dihydrofuran group of formula (II) below:

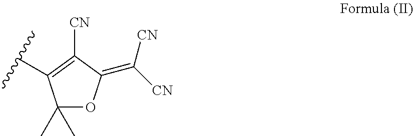

m is an integer between 2 and 10 inclusive.

The invention will be understood more clearly, and other advantages and characteristics thereof will emerge more clearly, on reading the explanatory description that follows.

The molecule used in the manufacturing processes of the invention is an n-type molecule with semiconducting properties, which is soluble in an organic solvent and air-stable.

This molecule has two essential characteristics: it is formed from a group noted as R bearing conjugated pi bonds comprising at least one aromatic ring, and at least two 2-dicyanomethylene-3-cyano-2,5-dihydrofuran groups are directly or indirectly bonded to this group R.

The aromatic group may furthermore be directly or indirectly bonded to electron-withdrawing groups, as in the prior art.

More specifically, the molecule of the invention has the formula (I) below:

(GEA)ₙ-R-(DCDHF)ₘ Formula (I)

In this formula, R represents the group bearing conjugated pi bonds comprising at least one inclusively 4- to 6-membered aromatic ring.

However, preferably, the group R comprises at least one phenylene or naphthalene or anthracene or perylene group, this aromatic ring optionally comprises at least one heteroatom chosen from N, O, P, S, Si and Ge.

It is also optionally substituted.

When it is substituted, the groups GEA, when present, and DCDHF, are bonded to these substituents.

Preferably, these substituents are unsaturated groups, which, in one particularly preferred embodiment, extend the pi conjugation of the aromatic ring(s).

In formula (I), DCDHF represents a 2-dicyanomethylene-3-cyano-2,5-dihydrofuran group of formula (II) below:

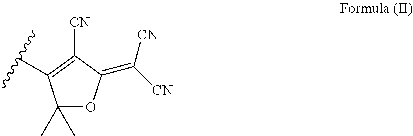

The molecule of formula (I) comprises at least two such

DCDHF groups, which means that m is an integer at least equal to 2.

Preferably, m is between 2 and 10 inclusive.

Also, in formula (I), GEA denotes an electron-withdrawing group, such as those known in the prior art.

These groups may or may not be present, which entails that n is an integer greater than or equal to zero.

Preferably, n is an integer between 0 and 10 inclusive and more preferably between 0 and 4 inclusive.

The group GEA may be, as has already been stated, any electron-withdrawing group known in the prior art, such as a fluorine atom; a cyano group (—C≡N); a fluorocarbon group of formula —(CF₂)ₓ—CF₃, with x preferably being between 0 and 17; a malononitrile group ═CH—(CN)₂; an anhydride group O═C—O—C═O; an imide group O═C—N—C═O, a nitro group —NO₂; a quaternary ammonium group N⁺R₁R₂R₃ with R₁, R₂ and R₃ representing, independently of each other, a C₁ to C₅ alkyl group, preferably a methyl, ethyl or butyl group, or a hydrogen atom; an ester group C(═O)—O—R₅, with R₅ preferably representing a group chosen from optionally substituted hydrocarbon-based groups, such as a methyl or isopropyl group; an amide group —C(═O)—N(R₇)—R₆, with R₆ and R₇ preferably representing a group chosen from optionally substituted hydrocarbon-based groups, such as a methyl or isopropyl group, and a hydrogen atom; or alternatively a sulfonyl group S(═O)₂(R₈), with R₈ preferably representing a group chosen from optionally substituted hydrocarbon-based groups, such as a methyl or isopropyl group, and a hydrogen atom.

Preferably, the GEA group is an electron-withdrawing group comprising a cyano or imide function or is a fluorine atom or a fluorocarbon chain.

Preferably, the molecule of formula (I) has at least one axis or plane of symmetry, advantageously C₂ symmetry, i.e. the molecule has an axis of symmetry perpendicular to the chain of the molecule.

In other words, a 180° rotation about the C₂ axis of symmetry gives the same molecule.

Preferred examples of a molecule of formula (I) are the molecules of formulae 1, 2, 3, 4 and 5 below:

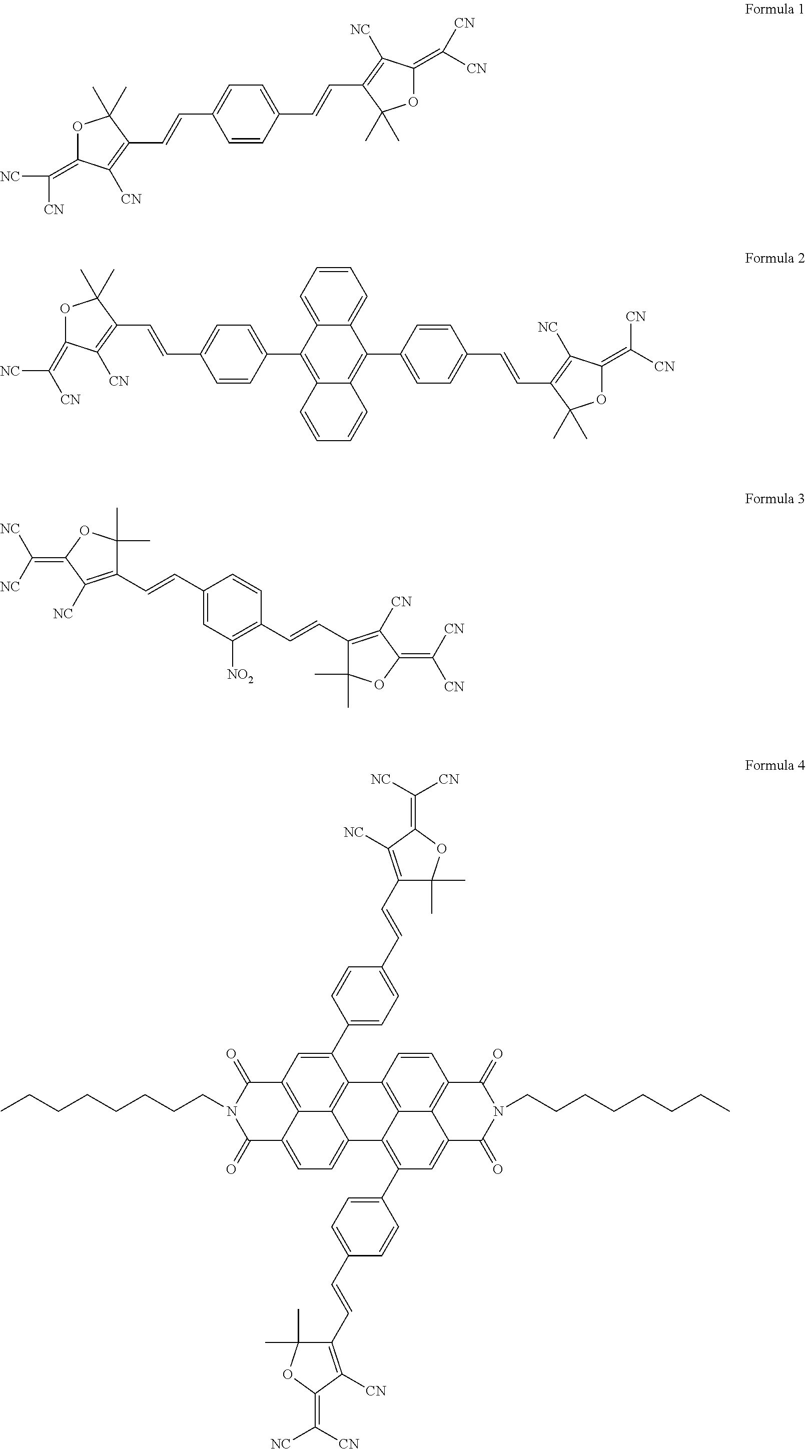

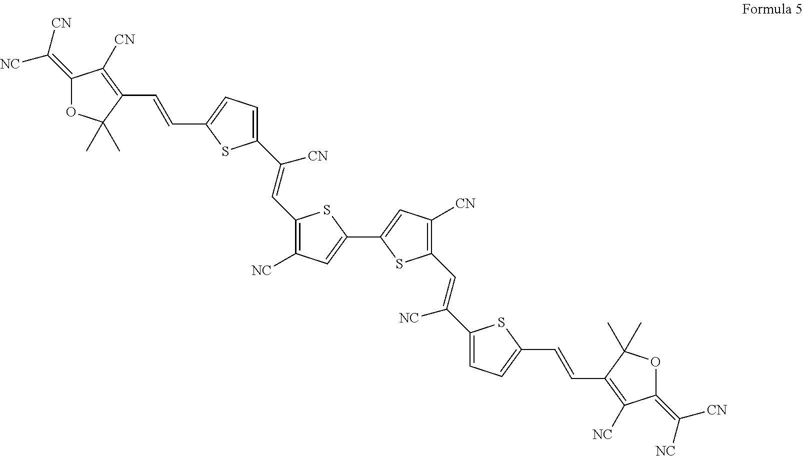

The molecules of the invention may be dissolved in at least one organic solvent.

As nonlimiting examples, this organic solvent is acetone, tetrahydrofuran, dimethylformamide, acetonitrile, benzonitrile, chloroform, ortho-dichlorobenzene or trifluoromethylbenzene.

The molecules of the invention have n-type semiconducting properties.

They may thus be used for the manufacture of an n-type organic semiconductor material and for the formation of a film made of an n-type organic semiconductor material when they are deposited on at least one surface of a substrate.

The deposition of these molecules may be performed via a liquid route, i.e. in solution, by deposition of drops, known as “drop casting”, optionally at a temperature close to the boiling point of the chosen solvent, or by spin coating or ink jet coating.

The molecule of the invention may also be deposited by flexography, by heliography or by formation of Langmuir and Langmuir-Blodgett films.

However, may also be deposited by vacuum sublimation.

On account of their n-type semiconducting properties, the molecules of the invention are advantageously used in a process for manufacturing a transistor.

More specifically, a process for manufacturing a transistor according to the invention comprises a step of depositing, onto at least one surface of a substrate, a solution comprising at least one molecule according to the invention.

Another process for manufacturing a transistor according to the invention comprises a step of using a molecule of formula (I) as described above.

The invention also relates to a transistor that comprises a layer made of an n-type organic semiconductor material comprising at least one molecule of formula (I), more preferably having one of the formulae 1 to 5 above.

The invention also proposes a molecule of formula (I) below:

(GEA)ₙ-R-(DCDHF)ₘ Formula (I)

in which:
GEA denotes an electron-withdrawing group,
n is a positive integer between zero and 10 inclusive,
R is a group bearing conjugated pi bonds comprising at least one inclusively 4-membered to 6-membered aromatic ring, this aromatic ring optionally comprising at least one heteroatom chosen from N, O, P, S, Si and Ge, and being optionally substituted,
DCDHF represents a 2-dicyanomethylene-3-cyano-2,5-dihydrofuran group of formula (II) below:

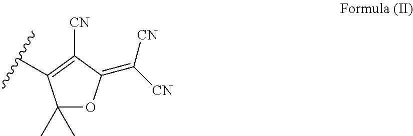

m is an integer between 2 and 10 inclusive, on condition that when n=0, then the molecule has the formula 1 or 2 below:

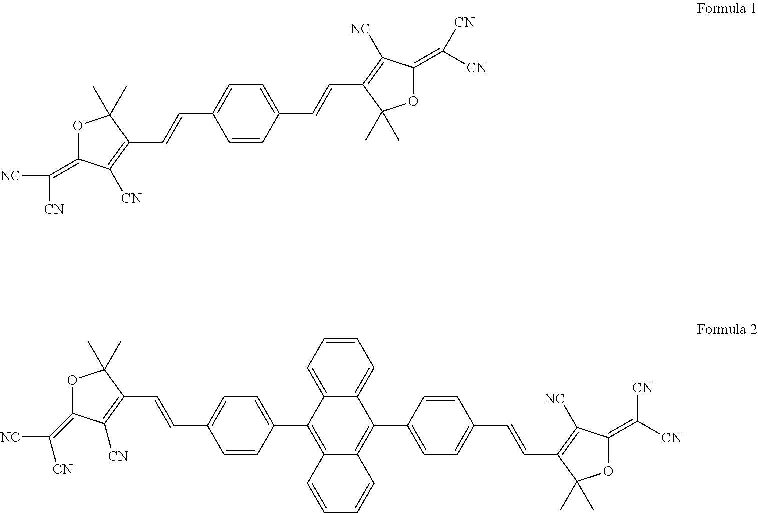

Preferably, this molecule is chosen from the molecules of formulae 1 to 5 below:

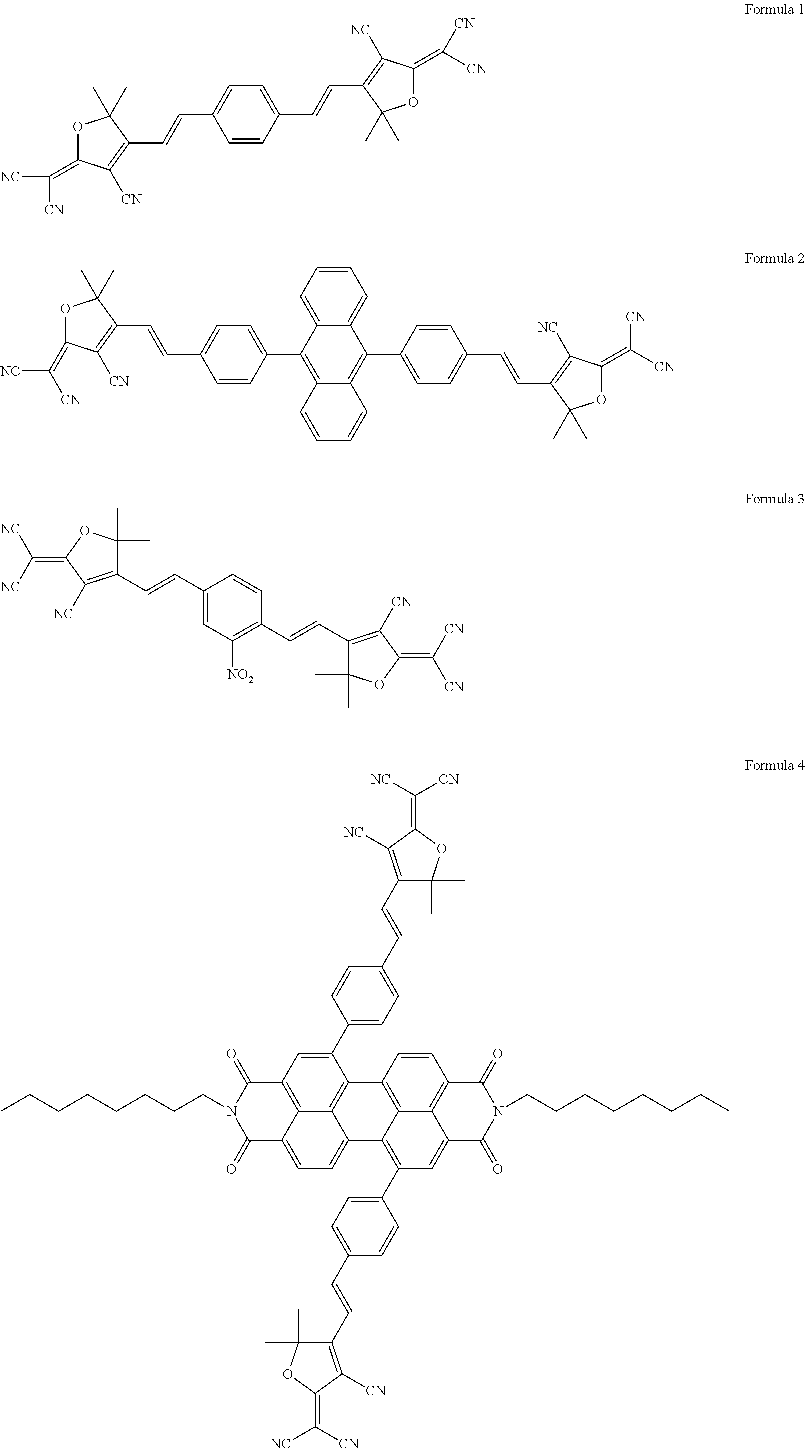

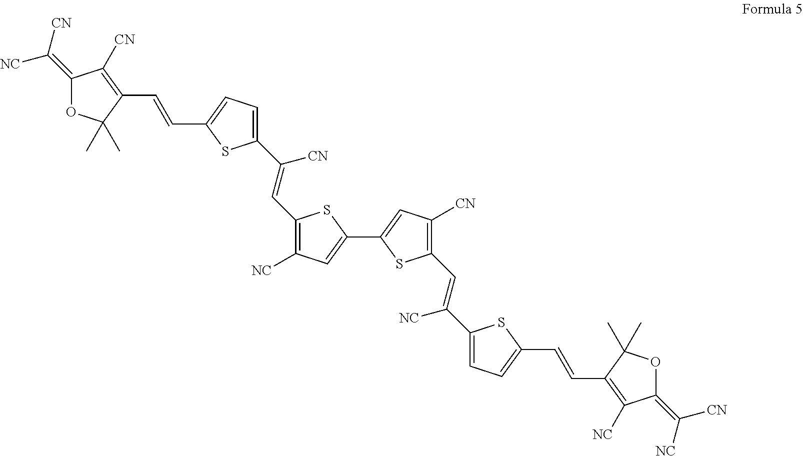

Several embodiments, which are given purely as nonlimiting illustrations, will now be described in order to explain the invention more clearly.

### EXAMPLE 1

### Synthesis of a Molecule According to the Invention

In the molecule synthesized in this example, the group R is a benzene group.

The molecule of the invention in this example does not contain an electron-withdrawing group GEA (n=0), but bears two groups DCDHF bonded to the benzene group via an unsaturated substituent (m=2):

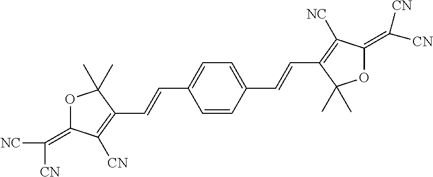

In this case, the DCDHF is first synthesized separately and then, by means of the acidity of the protons of the methyl group located in the a position relative to one of the cyanos, it is introduced via a reaction especially of Knoevenagel type on the terephthaldialdehyde. The double bond derived from the removal of a water molecule during the introduction of the DCDHF makes it possible to maintain extended conjugation throughout the molecule, which increases the electron delocalization.

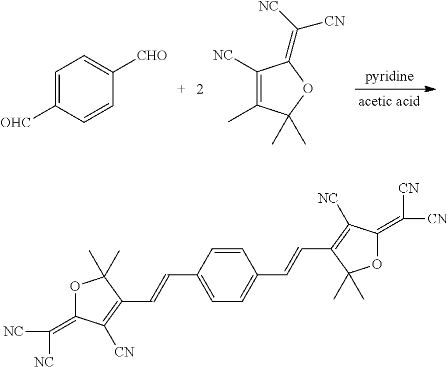

The product is obtained in the form of a red solid. Deposition by “drop casting” of a solution of this compound in dimethylformamide onto a transistor, irrespective of its geometry, results in the formation of crystals.

### EXAMPLE 2

### Synthesis of a Molecule According to the Invention

The molecule of the invention according to this example comprises a group R that is an anthracene and does not comprise an GEA group (n=0).

The molecule bears two DCDHF groups bonded to the aromatic group via an unsaturated substituent (m=2):

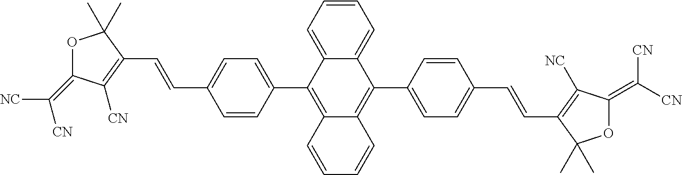

This compound is prepared by introducing two DCDHF molecules via a Knoevenagel-type reaction onto a bisaldehyde derivative of anthracene.

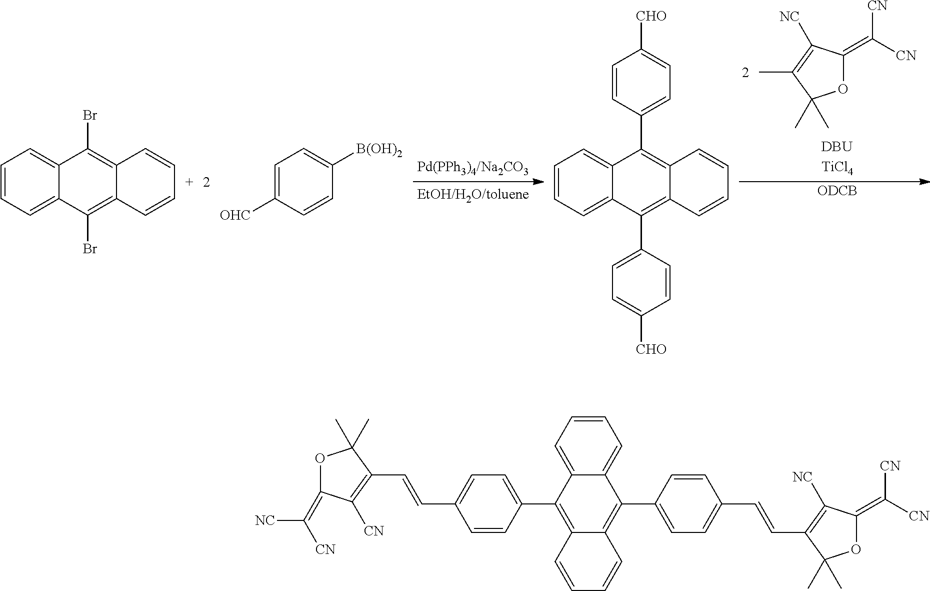

The bisaldehyde derivative of anthracene is formed in a first step by Suzuki coupling between 9,10-dibromoanthracene and 4-formylphenylboronic acid in the presence of a source of palladium and of a base in two-phase medium. Next, it reacts with two equivalents of DCDHF in ortho-dichlorobenzene in the presence of a base and a catalyst, titanium tetrachloride. The product is obtained in the form of a red solid. Deposition by drop casting of a solution of this compound in benzonitrile onto a transistor, irrespective of its geometry, results in the formation of crystals.

### EXAMPLE 3

### Synthesis of a Molecule According to the Invention

In the molecule synthesized in this example, the group R is a benzene group.

The molecule of the invention in this example contains an electron-withdrawing group GEA of nitro type (n=1) and bears two DCDHF groups bonded to the benzene group via an unsaturated substituent (m=2):

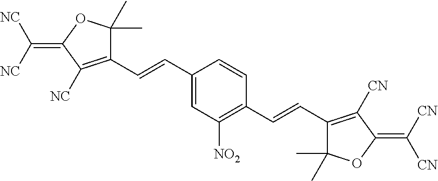

In this case, the DCDHF is first synthesized separately and then, by means of the acidity of the protons of the methyl group located in the a position relative to one of the cyanos, it is introduced via a reaction especially of Knoevenagel type onto 2-nitro-1,4-dibenzaldehyde, which is itself obtained by reduction of the corresponding diacid. The double bond derived from the removal of a water molecule during the introduction of the DCDHF makes it possible to maintain extended conjugation throughout the molecule, which increases the electron delocalization.

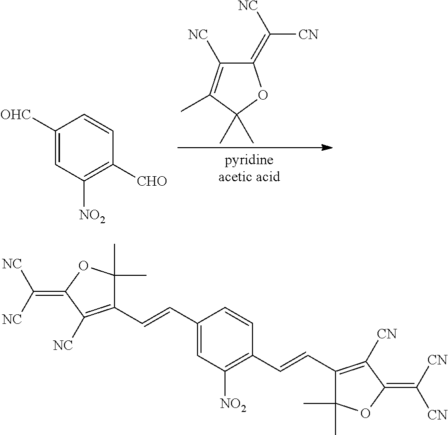

The product is obtained in the form of a brown solid. The deposition for the preparation of transistors is performed by “drop casting” of a solution of this compound in chloroform.

### EXAMPLE 4

### Synthesis of a Molecule According to the Invention

In the molecule of the invention according to this example, the group R is a perylene group bonded to two electron-withdrawing groups GEA comprising imide functions (n=2).

The molecule of this example bears two DCDHF groups bonded to the group R via an unsaturated substituent (m=2):

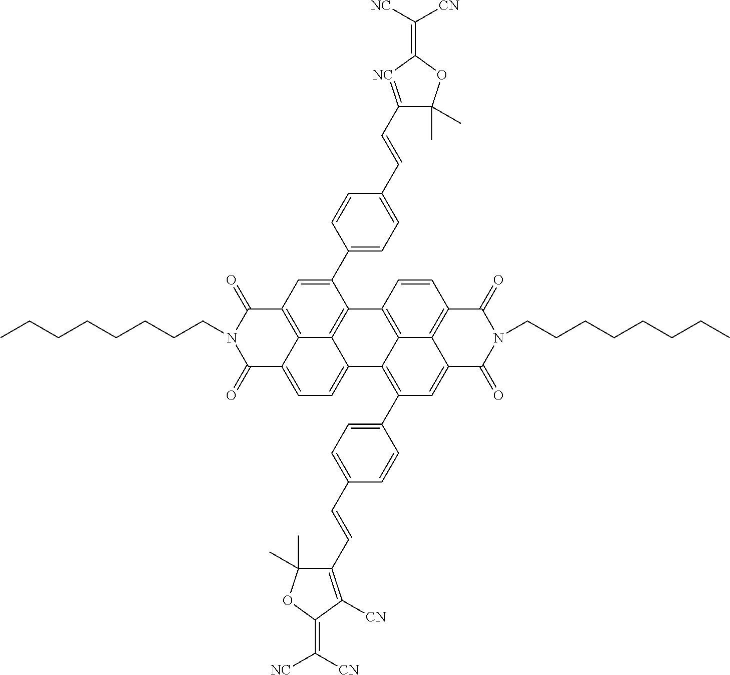

This compound is synthesized from perylene tetracarboxylic dianhydride.

Perylene tetracarboxylic diimide is first formed by reaction in imidazole at 180° C. with n-octylamine, and bromination thereof is then performed in refluxing dichloromethane.

At this stage, the 1,7 isomer may be isolated by chromatography optionally followed by successive recrystallizations from a dichloromethane/methanol mixture.

Finally, via Suzuki coupling with 4-formylphenylboronic acid and a Knoevenagel reaction with two equivalents of DCDHF under the conditions described in the preceding example, the product is obtained in the form of a red solid.

Deposition by depositing drops of a solution of this compound in dimethylformamide onto a transistor, irrespective of its geometry, results in the formation of crystals.

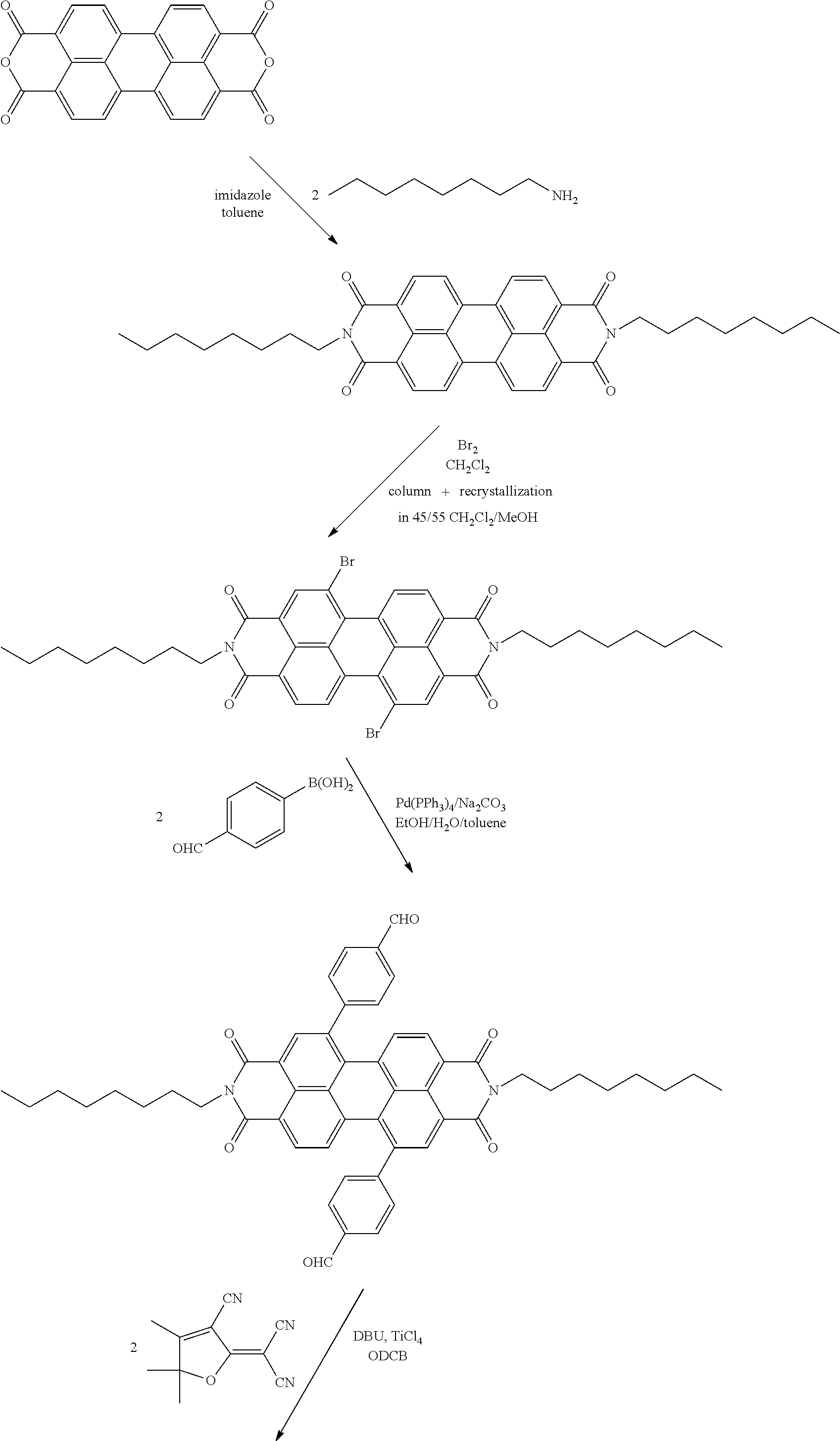

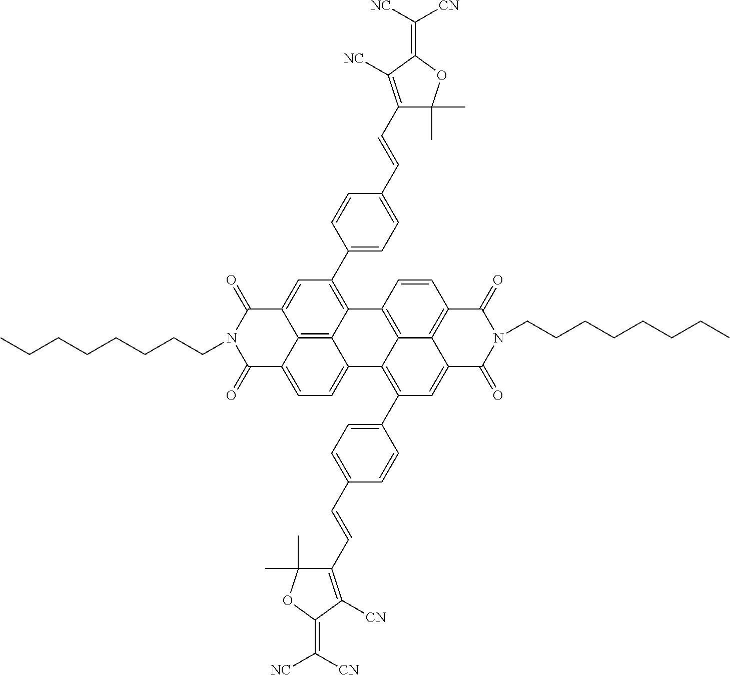

### EXAMPLE 5

### Synthesis of a Molecule According to the Invention

In the molecule synthesized in this example, the group R is a bithiophene group bearing two thiophenevinylene groups.

The molecule of the invention in this example contains four electron-withdrawing groups GEA of cyano type (n=4) and bears two DCDHF groups bonded to the bithiophene group bearing two thiophenevinylene groups via an unsaturated substituent (m=2):

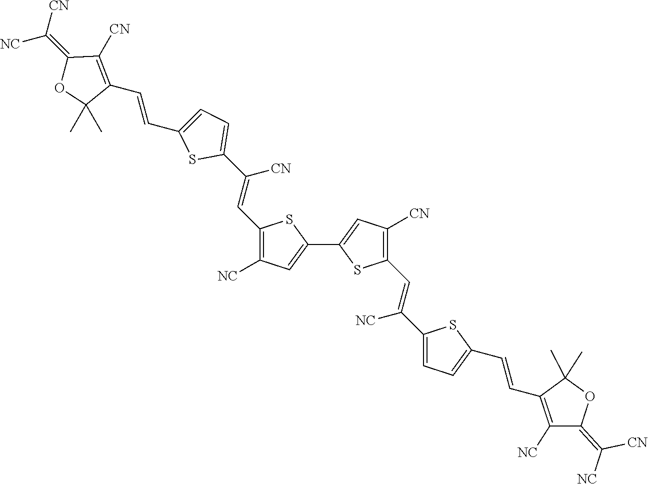

In this case, the 2-formyl-3-bromothiophene and 2-formyl-5-acetonitrilothiophene are first synthesized via a Vilsmeier-Haak reaction starting, respectively, with 3-bromothiophene and 2-acetonitrilothiophene. The DCDHF is then introduced onto the 2-formyl-5-acetonitrilothiophene via a reaction of Knoevenagel type.

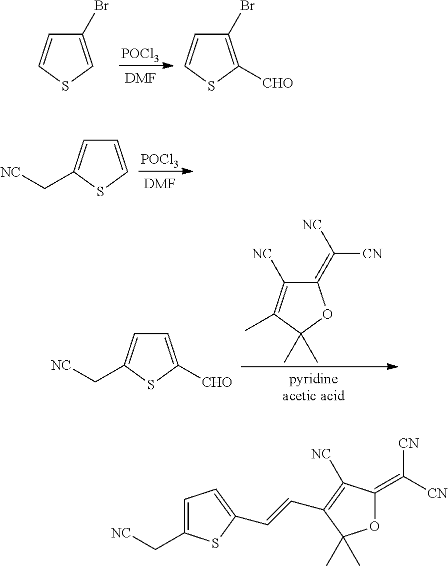

The compound formed is added via a reaction of Knoevenagel type to 2-formyl-3-bromothiophene and the bromine atom is then replaced with a cyano group by reaction with zinc dicyanide.

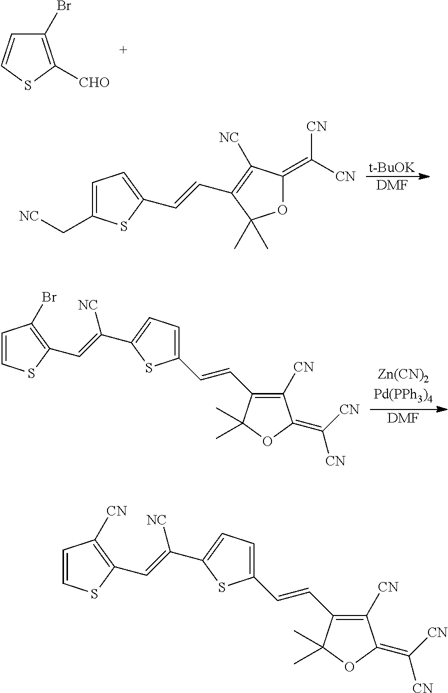

The derivative formed is then used in two different reactions. In one, it is brominated with N-bromosuccinimide, and in the other, a trimethyltin group is introduced.

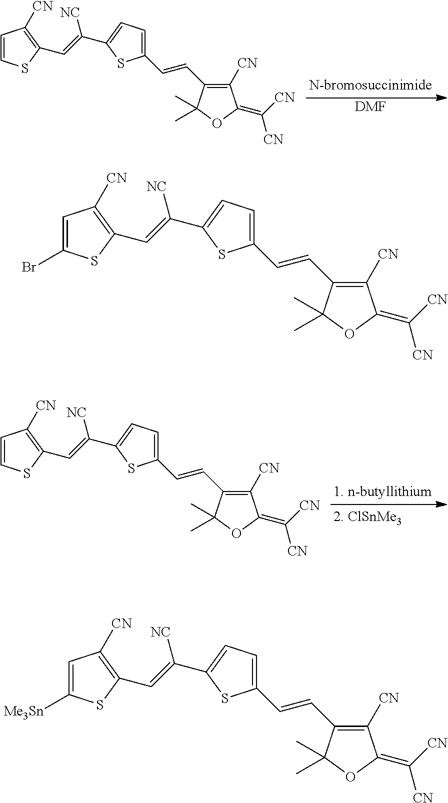

A final step involving a coupling reaction between the two compounds prepared in the presence of palladium(II) results in the desired product.

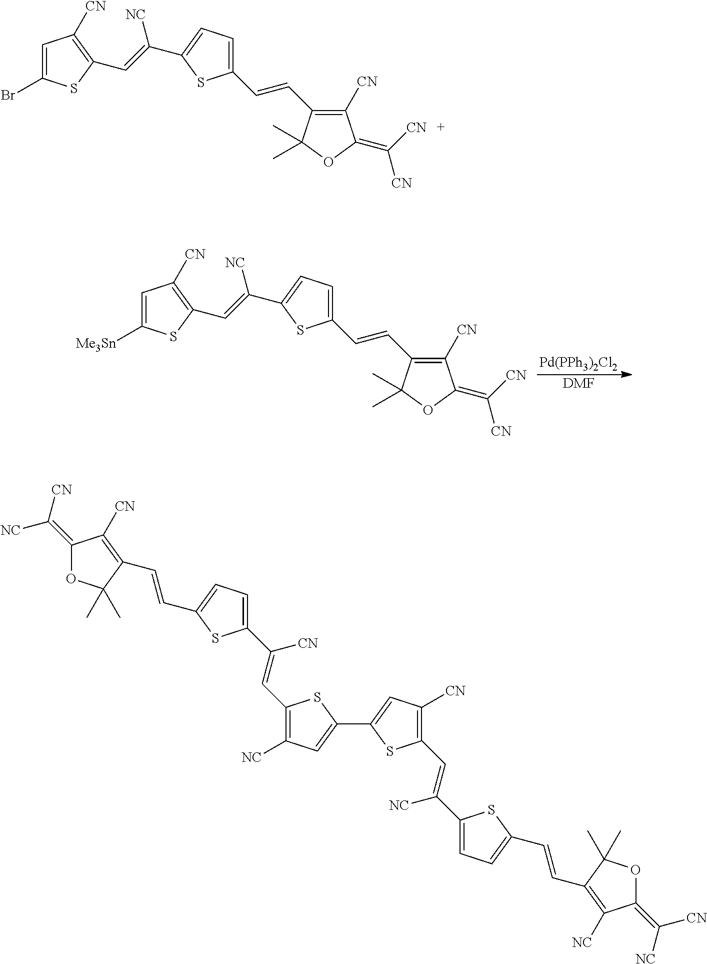

The conjugation is extended throughout the molecule, which increases the electron delocalization. The product is obtained in the form of a red solid. Deposition of the organic semiconductor is performed by “drop casting” of a solution of this compound in dimethylformamide.

### EXAMPLE 6

### Manufacture of Field-Effect Transistors with the Molecules of Examples 1 to 5 According to the Invention

Various field-effect transistors were manufactured and especially, by way of example, in the following manner (bottom-gate bottom-contact geometry):
sonication of a chip (1 cm×1 cm) comprising transistors bearing linear or interdigitated units (channel width W of 1000 to 10 000 pm and channel length L of 2 to 200 μm) in acetone for 3 minutes followed by rinsing with isopropanol and drying with argon,
UV/ozone treatment for 45 minutes,
formation of a self-assembled layer in the channels by dipping the chip in a solution of octyltrichlorosilane in toluene (10⁻³ M) for 4 hours at 80° C.,
deposition of the n-type semiconductor via one of the methods mentioned previously,
release of the electrodes from the transistors by means of an organic solvent belonging to the list presented in the description of the invention,
annealing at 100° C. for 20 minutes,
establishment of the electrical contacts and especially of the bottom-gate electrode,
annealing at 100° C. for 5 minutes.

### EXAMPLE 7

### Testing of the Transistors Obtained in Example 6

The field-effect transistors obtained in Example 6 were tested.

Their mobility and Iₒₙ/I_{off} ratio values, determined by electrical characterization in air, are as follows:
n-type semiconductor of Example 1: 3×10⁻⁵ cm². V⁻¹. s⁻¹ and 2×10²,
n-type semiconductor of Example 2: 3×10⁻⁴ cm². V⁻¹. s⁻¹ and 3×10³,
n-type semiconductor of Example 3: 1×10⁻⁴ cm². V⁻¹. s⁻¹ and 8×10²,
n-type semiconductor of Example 4: 6×10⁻³ cm². V⁻¹. s⁻¹ and 8×10⁴,
n-type semiconductor of Example 5: 8×10⁻⁴ cm². V⁻¹. s⁻¹ and 9×10²,

Examples 6 and 7 show that the molecules of the invention make it possible to obtain n-type semiconductor materials that have good conduction properties and that are manipulable in solution and in air.

1. A process for manufacturing a transistor, the process comprising:
contacting at least one surface of a substrate with a molecule of formula (I) below:
(GEA)ₙ-R-(DCDHF)ₘ Formula (I),
wherein:
GEA is an electron-withdrawing group;
n is a positive integer between zero and 10 inclusive;
R is a group comprising conjugated pi bonds comprising an optionally substituted 4-membered to 6-membered aromatic ring optionally comprising at least one heteroatom selected from the group consisting of N, O, P, S, Si, and Ge; and
DCDHF is a 2-dicyanomethylene-3-cyano-2,5-dihydrofuran of formula (II): 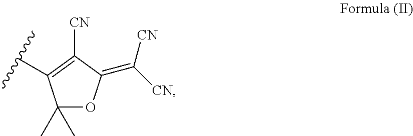
wherein m is an integer between 2 and 10 inclusive.

2. A process for manufacturing a transistor, the process comprising:
depositing, onto at least one surface of a substrate,a solution comprising a molecule of formula (I):
(GEA)ₙ-R-(DCDHF)ₘ Formula (I),
wherein:
GEA denotes is an electron-withdrawing group;
n is a positive integer between zero and 10 inclusive;
R is a group comprising conjugated pi bonds comprising an optionally substituted 4-membered to 6-membered aromatic ring optionally comprising a heteroatom selected from the group consisting of N, O, P, S, Si, and Ge; and
DCDHF is a 2-dicyanomethylene-3-cyano-2,5-dihydrofuran of formula (II): 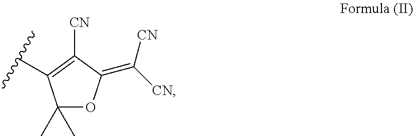
wherein m is an integer between 2 and 10 inclusive.

3. The process of claim 1, wherein, in formula (I), one the 4-membered to 6-membered aromatic ring is substituted with at least one unsaturated group.

4. The process of claim 1, wherein the molecule of formula (I) has at least one axis or plane of symmetry.

5. The process of claim 1, wherein, in formula (I), GEA is at least one selected from the group consisting of a fluorine atom, a cyano, a fluorocarbon, a malononitrile, an anhydride, an imide, a nitro, a quaternary ammonium, an ester, an amide, and a sulfonyl.

6. The process of claim 1, wherein, in formula (I), n is an integer between 0 and 4 inclusive.

7. The process of claim 1, wherein the molecule of formula (I) is selected from the group consisting of molecules represented by formulas 1 to 5:

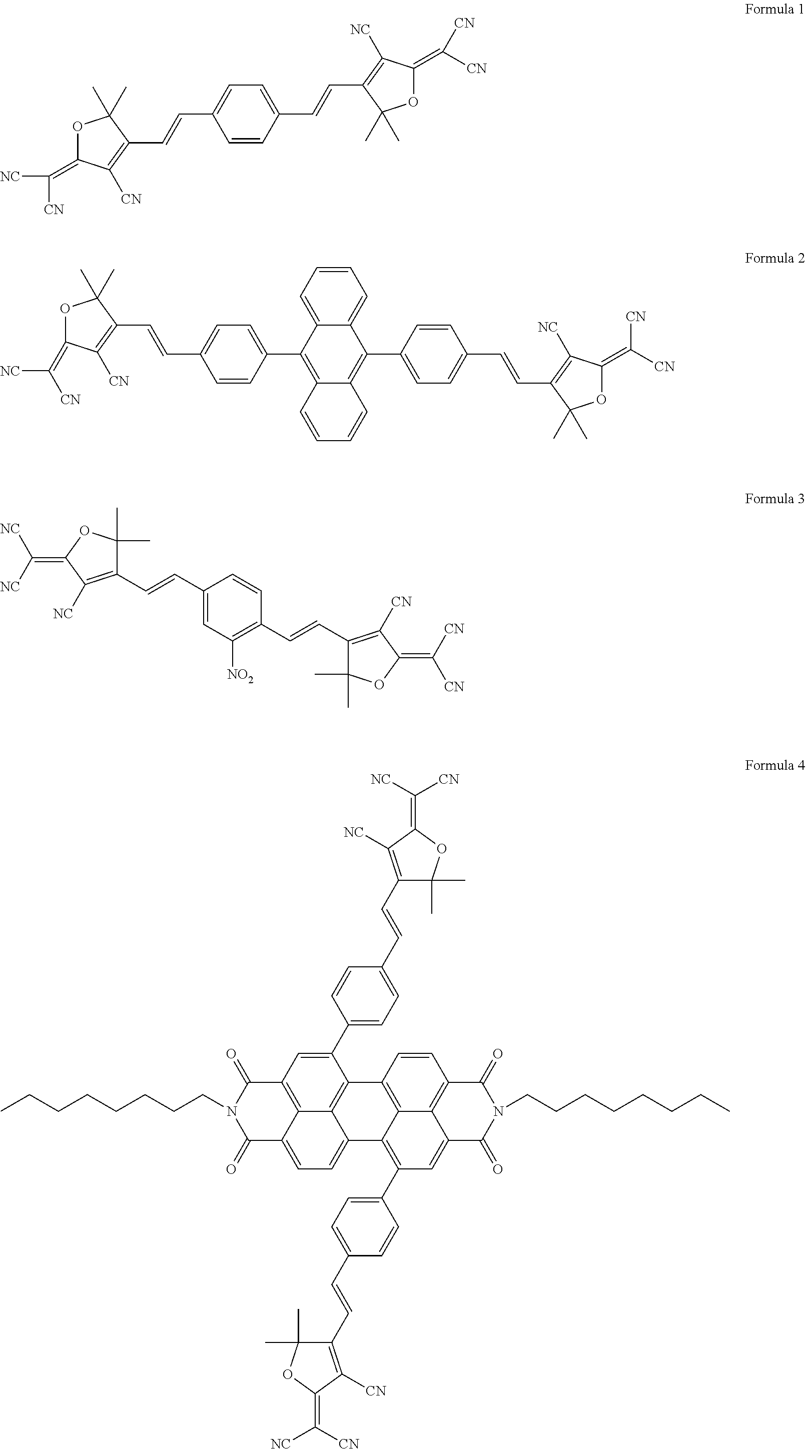

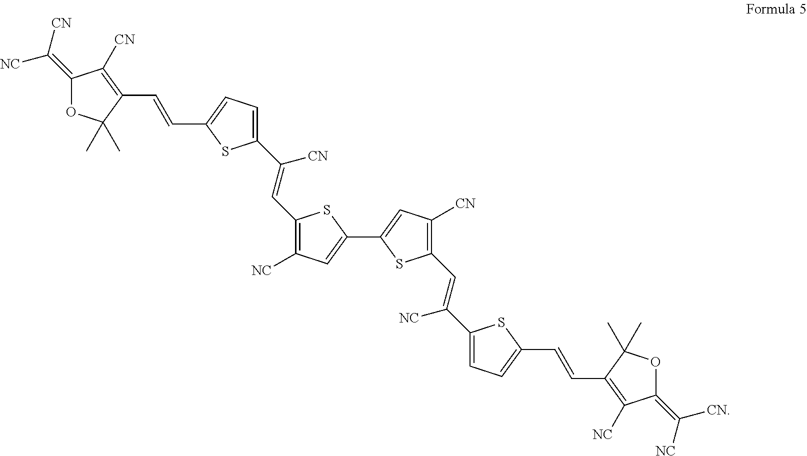

8. A molecule, of formula (I):
(GEA)ₙ-R-(DCDHF)ₘ Formula (I)
wherein:
GEA denotes is an electron-withdrawing group;
n is a positive integer between zero and 10 inclusive;
R is a group comprising conjugated pi bonds comprising an optionally substituted 4-membered to 6-membered aromatic ring optionally comprising at least one heteroatom selected from the group consisting of N, O, P, S, Si, and Ge; and
DCDHF is a 2-dicyanomethylene-3-cyano-2,5-dihydrofuran of formula (II): 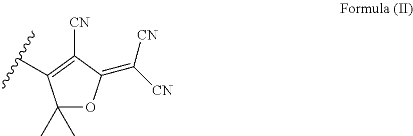
wherein m is an integer between 2 and 10 inclusive, and on condition that when n=0, the molecule is represented by formula 1 or 2:

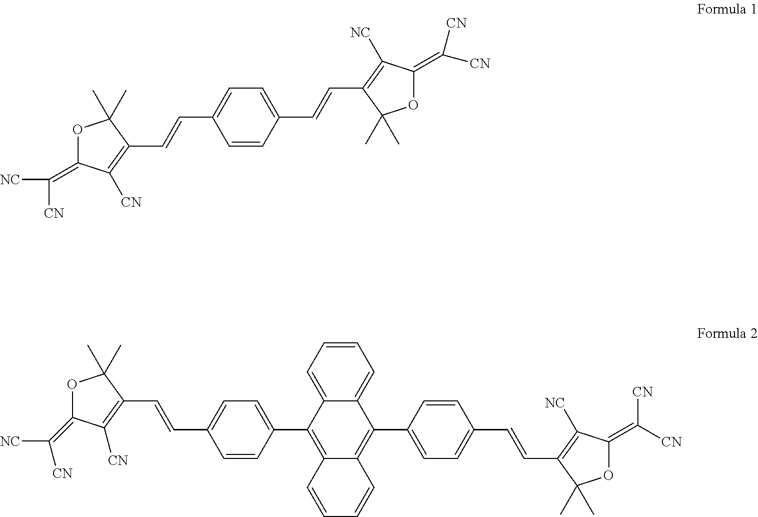

9. The molecule of claim 8, wherein the molecule is selected from group consisting of molecules represented by formulas 1 to 5:

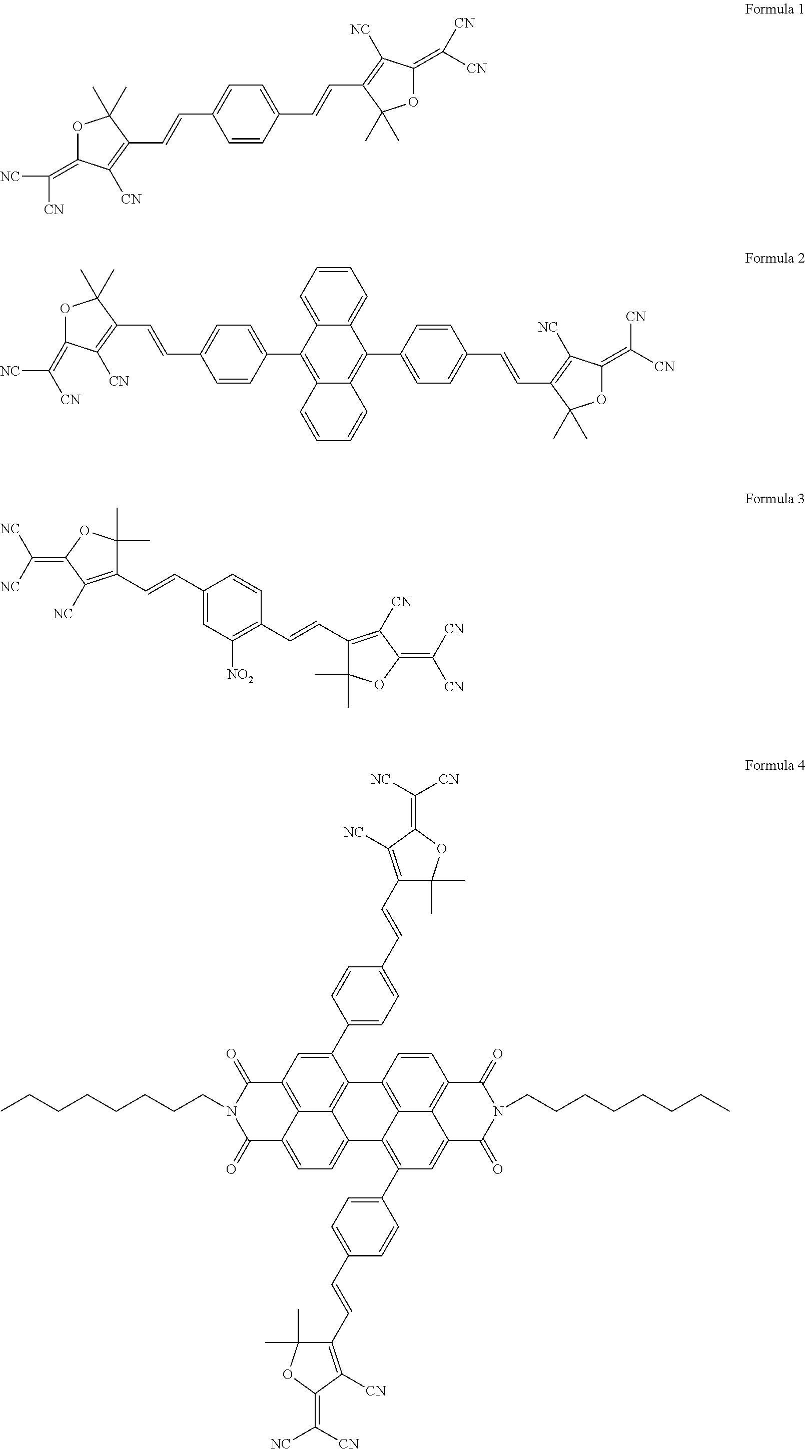

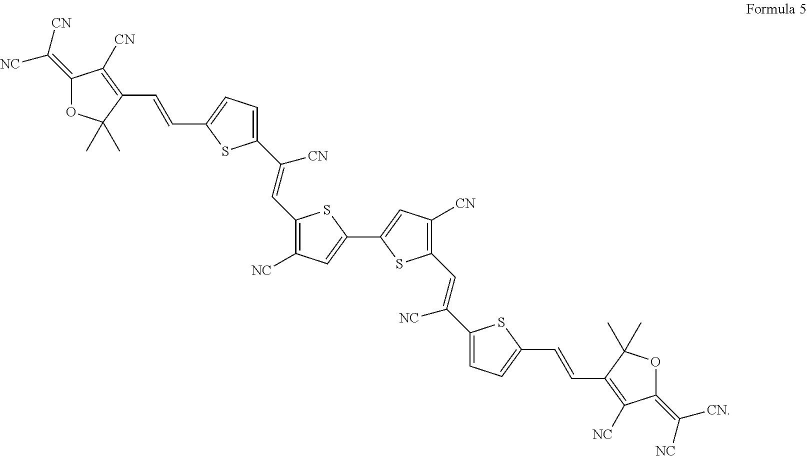

10. A transistor, comprising:
a layer comprising a molecule of formula (I):
(GEA)ₙ-R-(DCDHF)ₘ Formula (I),
wherein:
GEA is an electron-withdrawing group;
n is a positive integer between zero and 10 inclusive;
R is a group comprising conjugated pi bonds comprising an optionally substituted 4-membered to 6-membered aromatic ring optionally comprising at least one heteroatom selected from the group consisting of N, O, P, S, Si, and Ge; and
DCDHF is a 2-dicyanomethylene-3-cyano-2,5-dihydrofuran of formula (II): 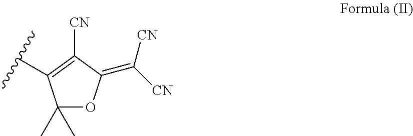
wherein m is an integer between 2 and 10 inclusive.

11. The process of claim 2, wherein, in formula (I), the 4-membered to 6-membered aromatic ring is substituted with at least one unsaturated group.

12. The process of claim 2, wherein the molecule of formula (I) has at least one axis or plane of symmetry.

13. The process of claim 1, wherein the molecule of formula (I) has C₂ symmetry.

14. The process of claim 2, wherein the molecule of formula (I) has C₂ symmetry.

15. The process of claim 2, wherein, in formula (I), GEA is at least one selected from the group consisting of a fluorine atom, a cyano, a fluorocarbon, a malononitrile, an anhydride, an imide, a nitro, a quaternary ammonium, an ester, an amide, and a sulfonyl.

16. The process of claim 3, wherein, in formula (I), GEA is at least one selected from the group consisting of a fluorine atom, a cyano, a fluorocarbon, a malononitrile, an anhydride, an imide, a nitro, a quaternary ammonium, an ester, an amide, and a sulfonyl.

17. The process of claim 4, wherein, in formula (I), GEA is at least one selected from the group consisting of a fluorine atom, a cyano, a fluorocarbon, a malononitrile, an anhydride, an imide, a nitro, a quaternary ammonium, an ester, an amide, and a sulfonyl.

18. The process of claim 2, wherein, in formula (I), n is an integer between 0 and 4 inclusive.

19. The process of claim 3, wherein, in formula (I), n is an integer between 0 and 4 inclusive.

20. The process of claim 4, wherein, in formula (I), n is an integer between 0 and 4 inclusive.
